(19) United States
(12) Patent Application Publication
(10) Pub. No.: US 2005/0020814 A1
(43) Pub. Date: Jan. 27, 2005

(54) Method for renaturating proteins

(76) Inventors: Rainer Rudolph, Halle/Saale (DE); Hauke Lilie, Halle/Saale (DE); Uta Raue, Merseburg (DE)

Correspondence Address: JENKINS & WILSON, PA, 3100 TOWER BLVD, SUITE 1400, DURHAM, NC, 27707, US

(21) Appl. No.: 10/865,135

(22) Filed: Jun. 10, 2004

Related U.S. Application Data
(63) Continuation of application No. PCT/EP02/13732, filed on Dec. 4, 2002.

(30) Foreign Application Priority Data
Dec. 14, 2001 (DE) 10161577.9

(51) Int. Cl. C07K 14/00

(52) U.S. Cl. 530/350; 530/412

(57) ABSTRACT

The present invention relates to the use of substituted imidazolium salts for the renaturation, for the decrease of aggregation and/or for the increase of the thermal stability of proteins as well as to a method for the renaturation of proteins, for the decrease of the aggregation and/or for the increase of the thermal stability of proteins as well as to renatured proteins prepared by means of the method according to the invention. In the method according to the invention, the proteins to be treated are contacted with a liquid renaturation medium containing substituted imidazolium salts.

The invention relates to the use of substituted imidazolium salts for the renaturation, for the decrease of aggregation and/or for the increase of the thermal stability of proteins as well as to a method for the renaturation, for the decrease of aggregation and/or for the increase of the thermal stability of proteins as well as to renatured proteins prepared by means of the method according to the invention.

In the present context, the renaturation of proteins is considered to be the recovery or the restoration of the biological activity and/or of an to a high extent correct folding of proteins. The problem of protein renaturation is of high importance in biotechnology. Due to the developments achieved in molecular biology it is possible to clone proteins starting with the coding sequences thereof and to produce them in host organisms in a recombinant manner. In these cases, however, the recombinant proteins often precipitate in the form of biologically inactive aggregates or inclusion bodies. The protein contained in these inclusion bodies must subsequently be transformed into the biologically active form by means of in vitro renaturation.

This renaturation is a very complex structural biological process and often results in only very small yields of native protein. Therefore, even increases in the yield of renatured protein of a few percent would provide a substantial improvement and an economic advantage.

In the in vitro folding of inclusion body proteins there has often been observed a kinetic competition between correct folding and unproductive side reactions such as malfolding and aggregation. The aggregation processes are mostly based on hydrophobic interactions of polypeptide chains which are largely unfolded or of non-native, partially folded intermediates. Irrespective of their molecular basis these aggregation events have a reaction order of >2. Consequently, the rate of these unproductive aggregation reactions increases with an increase in the concentration of the unfolded polypeptide chain. Since correct folding events mostly are governed by first order reactions, this means that the aggregation reactions predominate over correct folding if the concentration of denatured protein is increased. For this reason, in vitro folding reactions are often performed at a very low protein concentration. In addition, physical parameters such as pH, ionic strength, redox system, and temperature must be optimized for protein renaturation. In many cases, however, even these measures does not bring about the desired success.

Furthermore, it is known for a long time that the addition of buffers such as urea or guanidinium in a non-denaturing concentration can have a positive impact on the efficiency of folding. As an alternative to guanidinium or urea also other chaotropic substances have been used in in vitro folding processes, for example alkyl urea, or organic co-solvents such as carboxylic acid amides or alkylated amines.

In the case of the renaturation of the human plasminogen activator from inclusion bodies of E. coli it has been observed that the yield of in vitro folding can be enhanced by an addition of L-arginine. Further studies have revealed that L-arginine can serve to improve the efficiency of folding also in the in vitro folding of other proteins such as antibody fragments or immunotoxins. Although arginine contains a guanidino group it is not as destabilizing for the structure as guanidinium. Accordingly, arginine is thought to increase the solubility of unfolded polypeptide chains or of folding intermediates without significantly destabilizing the native protein structure. Other additives which increase the efficiency of in vitro folding are highly concentrated Tris buffers, polyethylene glycol or detergents. Improved yields of folding have also been achieved by using mixed micelles (for example of Triton X 100 and phospholipids). These mixed micelles contain both polar and apolar groups which presumably suppress the aggregation of folding intermediates by interacting with them.

The publication Amersham Pharmacia Biotech; Data File: Affinity Chromatography; HiTrap Chelating 1 ml and 5 ml; 18-1134-78 AB, page 1-6; 05/2000 describes a renaturation of a His₆-tagged protein on a Ni²⁺-NTA column. The renaturation buffer contains 20 mM of imidazole. This concentration of imidazole prevents unspecific interactions between proteins and the column matrix but does not result in any renaturation. On the contrary, imidazole is known as a denaturing agent.

Yeh et al., Hierachical folding of cytochrome c; Nature Struct. Biol. 7, page 443-445, 2000, describes that imidazole can accelerate the folding kinetics of the cytochrome c protein. The intramolecular interaction of His33 of the protein with its covalently bound ligand was suppressed by imidazole. Imidazole in higher concentrations essentially has a denaturing effect on proteins.

Despite a plurality of protocols for the renaturation of proteins mentioned in the literature the industrial utilization of this technique is not very well advanced. The reasons for this fact are based both on the technical and on the biochemical difficulties. Since even under optimal conditions the renaturation of proteins can only be achieved in the presence of very low protein concentrations an industrial production requires the work-up of very high renaturation volumes. Moreover, even under optimal conditions the efficiency of renaturation of many proteins is low.

Therefore it is an object of the invention to identify substances which enhance the efficiency of protein folding in vitro and to provide a method for the efficient renaturation of proteins in higher yields as those obtained to date.

This object is achieved according to the invention by using substituted imidazolium salts for the renaturation, for the decrease of aggregation and/or for the increase of the thermal stability of proteins as well as by a method for the renaturation, for the decrease of aggregation and/or for the increase of the thermal stability of proteins wherein the proteins to be treated are contacted with a liquid medium containing substituted imidazolium salts. The present invention also relates to proteins prepared by using the method according to the invention.

The preferred use according to the invention is the use for the renaturation of proteins.

According to the invention, the decrease of aggregation is intended to mean a decrease or even a substantial prevention of the aggregation of proteins which usually occurs during prolonged storage periods in liquid media and which is accompanied by a loss or a reduction in biological functionability.

According to the invention, increase of the thermal stability means that the biological activity or the correct protein folding, respectively, are maintained over a prolonged period of time even at temperatures which can be by far higher than room temperature.

The imidazolium salts used according to the invention preferably are ionic liquids at room temperature. If these compounds are not liquid at room temperature the imidazolium salts according to the invention should at least be present in a liquid form and/or be soluble in the liquid medium under the conditions of treatment. The imidazolium salts used in the frame of the present invention have a positive charge of the organic component associated with the ring system, which positive charge is usually or preferably delocalized within the imidazolium ring.

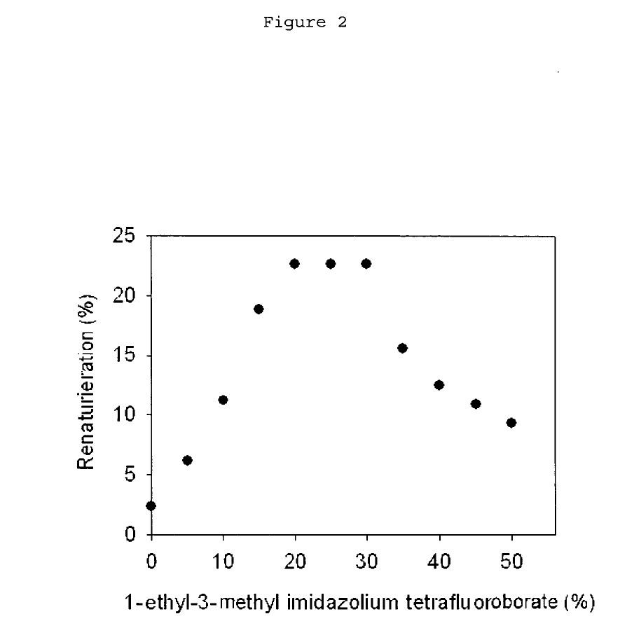

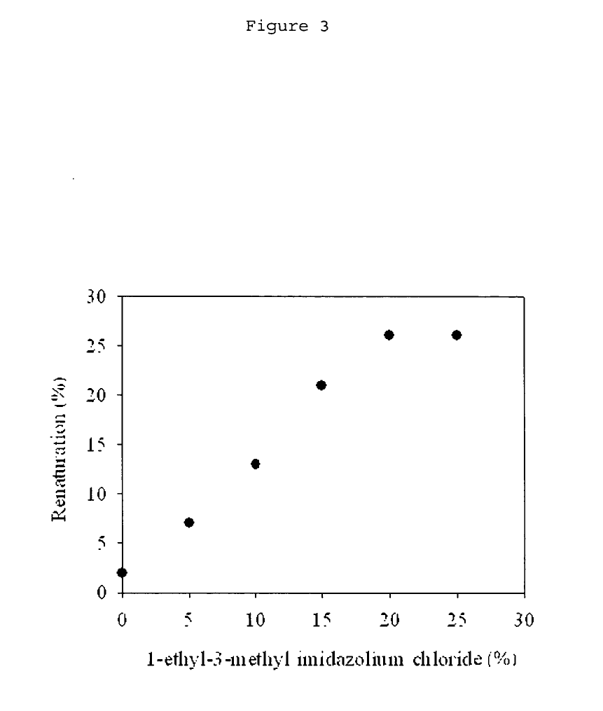

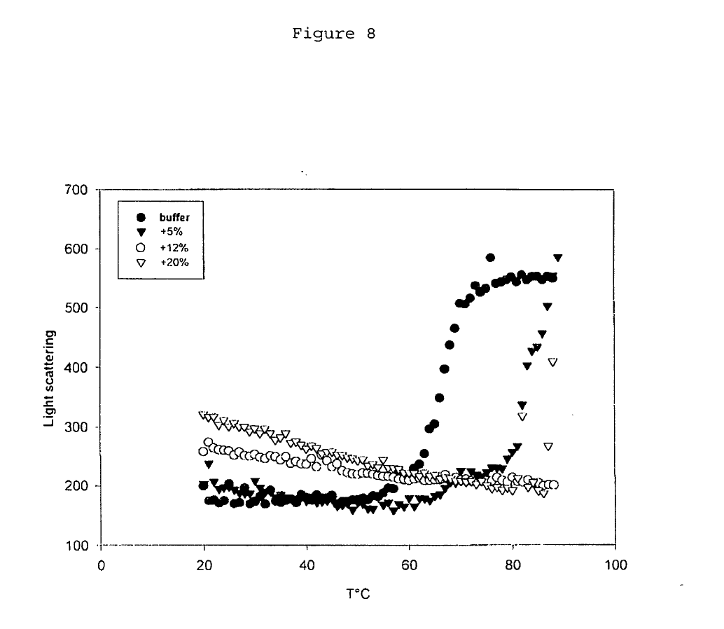

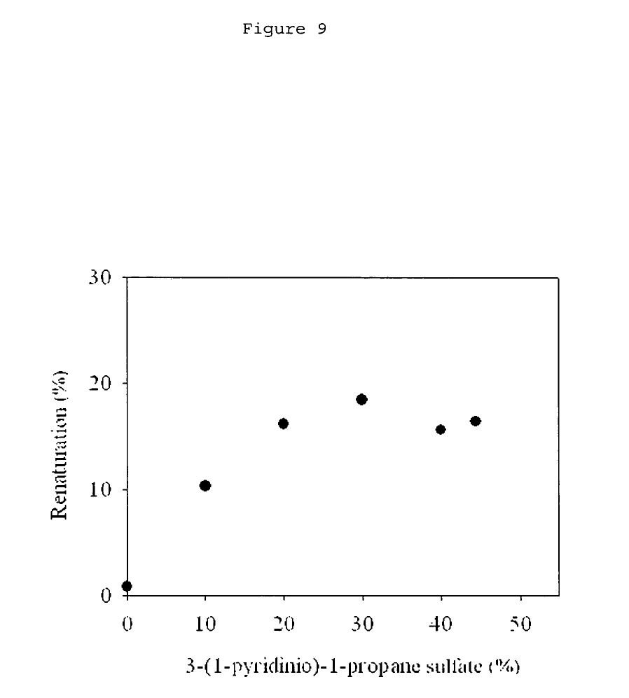

The method is based on the surprising discovery that substituted imidazolium salts suppress the aggregation of proteins and enhance the efficiency of renaturation. Up to now, these organic salts have only been used as solvents in organic synthesis and in two-phase catalysis whereas an effect on structure-forming processes in biochemistry has been unknown so far. It has been described also for other organic salts that they allow for a more efficient renaturation of proteins, for example for 3-(1-pyridinio)-1-propane sulfate or trigonelline hydrochloride (WO 99/18196). These substances for example have no positive charge delocalized within the heterocyclic ring system. In a direct comparison of their effect on the renaturation of proteins the imidazolium derivatives of the present invention show a clearly higher efficiency (see also the working examples as well as FIGS. 2 and 3 together with FIGS. 8 and 9).

It is preferred that the imidazolium rings of the imidazolium salts of the present invention are substituted by alkyl, alkenyl, aryl and/or aralkyl groups which may themselves be substituted by functional groups such as by groups containing nitrogen, sulfur and/or phosphorous wherein different oxidation states are possible. Preferred examples of these functional groups according to the invention are: amine, carboxyl, carbonyl, aldehyde, hydroxy, sulfate, sulfonate and/or phosphate groups.

According to the invention one or both of the N atoms of the imidazolium ring can be substituted by identical or different substituents. Preferably both nitrogen atoms of the imidazolium ring are substituted by identical or different substituents.

It is also possible or preferred according to the invention that the imidazolium salts are additionally or exclusively substituted at one or more of the carbon atoms of the imidazolium ring.

Preferred as the substituents are C₁-C₄ alkyl groups such as methyl, ethyl, n-propyl, isopropyl, n-butyl and/or isobutyl groups, particularly preferred ethyl and/or methyl groups. Substituents which are also preferred are C₂-C₄ alkenyl groups such as ethylene, n-propylene, isopropylene, n-butylene and/or isobutylene. With these C₁-C₄ alkyl groups and C₂-C₄ alkenyl groups the imidazolium compounds according to the invention have a water solubility which is particularly useful according to the invention and which decreases with longer alkyl and alkenyl chains. However, the water solubility can also be improved by solubility-promoting substituents on the alkyl or alkenyl chains themselves, for example by sulfate, sulfonate, amino or phosphate groups. According to the invention, also alkyl and alkenyl substituents having more than 4 C atoms are comprised wherein for example also C₅-C₁₀ alkyl or alkenyl substituents are still preferred. These C₅-C₁₀ alkyl or alkenyl groups are further preferred if they have one or more other substituents such as phosphate, sulfonate, amino and/or phosphate groups at their alkyl and/or alkenyl groups.

As the aryl substituents are preferred according to the invention mono- and/or bicyclic aryl groups, phenyl, biphenyl and/or naphthalene as well as derivatives of these compounds which carry hydroxy, sulfonate, sulfate, amino, aldehyde, carbonyl and/or carboxy groups. Examples of preferred aryl substituents are phenol, biphenyl, biphenol, naphthalene, naphthalene carboxylic acids, naphthalene sulfonic acids, biphenylols, biphenyl carboxylic acids, phenol, phenyl sulfonate and/or phenol sulfonic acids.

Preferred aralkyl groups are benzyl or substituted benzyl groups.

It is particularly preferred that the imidazolium ring has a methyl group at at least one of its N atoms.

The imidazolium salts used according to the invention are preferably liquids, i.e. preferably they are liquids which are ionic at room temperature (about 25° C.). However, also imidazolium salts can be used which are not liquid at room temperature but which then should be present in a liquid form or should be soluble in the liquid medium, respectively, under the renaturation conditions.

Imidazolium salts differ in their properties clearly from imidazole. Particularly, imidazolium salts are hydrophilic whereas imidazole is hydrophobic and has a denaturing effect on proteins.

As the anionic counter ions for the substituted imidazolium rings according to the invention halogens and halogen containing ions are especially useful. Particularly preferred in this respect are chloride and tetrafluoroborate. Further preferred are phosphate, sulfate and isocyanate.

Imidazolium salts which are more preferably used according to the invention are: 1-ethyl-3-methyl imidazolium tetrafluoroborate, 1-ethyl-3-methyl imidazolium chloride, 1-butyl-3-methyl imidazolium tetrafluoroborate.

Preferably according to the invention the following protein classes and/or proteins can be renatured, the thermal stability thereof can be enhanced and/or the aggregation thereof decreased, or the following proteins and/or protein classes can be preferably employed in the method according to the invention, respectively:
proteases, preferably serine proteases, particularly thrombin, factor Xa, caspases, cathepsins, trypsin and chymotrypsin, cysteine proteases, acidic proteases such as pepsin or rennin, metalloproteinases such as thermolysin; protease inhibitors, preferably pepstatins, antipain, chemostatins, elastinal, leupeptins, bestatin, antithrombin III;
DNA binding proteins, preferably transcription factors, particularly NF kappa B and members of the jun, fos, krox, myc, E2F families, viral T antigens;
viral proteins, e.g. viral envelope proteins, capsid proteins, viral proteases, polymerases and/or T antigens;
phosphatases;
protein kinases, preferably tyrosine kinases and/or serine kinases;
proteins of the immunoglobulin superfamily, e.g. antibodies and fragments thereof; growth factors, e.g. epidermal growth factor (EGF), erythropoietin, fibroblast growth factor (FGF), insulin-like growth factors I and II (IGF I and IGF II), interleukin-2 (IL-2), nerve growth factor (NGF), transforming growth factor β (TGF-β) and/or thrombocyte growth factor (PDGF),
as well as proteins and fragments derived from these proteins.

According to the invention, disulfide-free and disulfide-bridged proteins can be renatured and/or the thermal stability thereof can be improved and/or the aggregation thereof can be prevented or decreased, respectively. Particularly preferred, multidomain proteins and complex disulfide-bridged proteins can be treated such as e.g. lysozyme, rPA (recombinant plasminogen activator, trade name replase), alpha glucosidase, antibodies and fragments derived therefrom and/or growth factors. These proteins/protein classes merely serve as examples, however, the invention is not limited to this enumeration.

As the liquid medium in the method according to the invention for the renaturation, for the decrease of aggregation and/or for the increase of the thermal stability of proteins there is preferably used a buffer system which is suitable for the protein to be treated. Tris, Hepes, Mes, Mops, acetate, glycine and/or phosphate are preferably used as the buffer substances, the buffer concentration in the liquid medium preferably being 10-1000 mM, more preferably 5-200 mM, also preferred 10-200 mM and further preferred 10-50 mM. The pH of the buffer system preferably is 4-11, further preferred 7.5-10.5. In the case of lysozyme the pH value during renaturation is preferably about 8, and for the renaturation of rPA the pH value preferably is about 10.5. For other proteins to be renatured the buffer composition parameters may be adapted and optimized individually to obtain a maximal yield of renatured protein.

In the method according to the invention renaturation of proteins the contacting of the proteins to be renatured or to be treated, respectively, is preferably performed by diluting, dialyzing and/or diafiltrating the protein to be treated with the liquid medium. Principally, a buffer exchange of the denatured protein into the renaturation buffer should be assured for the renaturation.

During contacting and particularly during the renaturation, the protein concentration of the protein to be treated preferably is 5-500 μg/ml, preferably 10-100 μg/ml, still more preferably about 50-100 μg/ml. These values can be adjusted by those skilled in the art with respect to the protein to be renatured or to be treated, respectively, considering the solubility properties of the respective protein.

The method according to the invention is useful for the renaturation of disulfide-free and disulfide-bridged proteins. Regarding disulfide-free proteins, these are preferably contacted with the renaturation medium which contains substituted imidazolium salts in the presence of a reduction agent such as DTT, DTE and/or cysteine, preferably in a concentration of 1-10 mM.

Regarding the renaturation of disulfide-bridged proteins, these are preferably contacted in the presence of a redox system composed of reduced and oxidized thiol substances such as DTT, DTE, glutathione, cysteine, mercaptoethanol, preferably in a concentration of 1-10 mM. In these cases, the concentration ratios of reduced substances to oxidized substances (“reduced:oxidized”) preferably are 1:10 to 20:1, preferably 1:5-10:1, further preferred 1:1 to 5:1.

During contacting and particularly during the renaturation the concentration of substituted imidazolium salts preferably is 5-95 vol. %, further preferred 5-50 vol. %, further preferred 10-40 vol. % based on the renaturation medium. Also in these cases the optimum concentration of each of the proteins may be determined by simple experimentation. Usually, the addition of substituted imidazolium salts in a ratio of 10-40 vol. % provides the best renaturation yields. As already mentioned above, however, the concentration can be up to 95 vol. % depending on the respective imidazolium compounds used.

In terms of molarities the above-mentioned volume percentages of 10-40 vol. % correspond to approximately 0.5-3 M: in the case of 1-ethyl-3-methyl imidazolium chloride this refers to exactly 0.68-2.73 M. In terms of molarities, therefore, it is preferred according to the invention that the molarities of substituted imidazolium salts are about 0.25-5 M, further preferred 0.25-3.5 M and most preferably about 0.5-3 M based on the renaturation medium.

Generally, the period of renaturation preferably is: 0.1-100 h, further preferred 1-50 h, still more preferably about 2-20 h.

The renaturation preferably takes place at low temperatures of 0-37° C., preferably 5-15° C. since at higher temperatures the aggregation reactions increase.

As a parameter for optimizing the renaturation the biological activity of the protein and the aggregation behavior can be preferably measured over the course of the process.

In another embodiment of the method according to the invention the protein may be added to the renaturation buffer at several successive times in a pulsed manner or in a continuous manner.

In conclusion, the method according to the invention is suitable in a particularly advantageous manner for the renaturation of proteins. In this respect it is further preferred that besides the substituted imidazolium salts the liquid medium for the renaturation of proteins contains other known renaturation substances or substances which promote renaturation such as for example urea, guanidinium, L-arginine, alkyl urea, carboxylic acid amides, alkylated amines, Tris buffers, polyethylene glycol and/or detergents.

In the following the present invention will be described with respect to working examples. Reference is made in this respect to the following Figures:

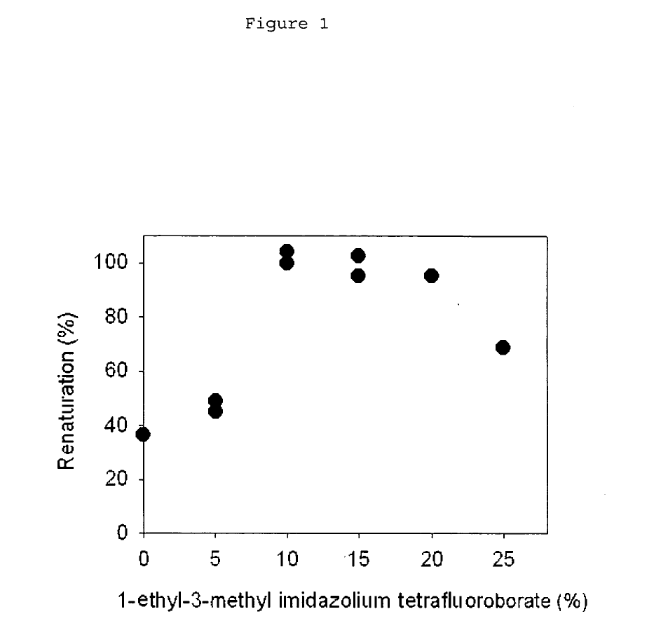

FIG. 1: shows the relationship between the yield of renaturation in % for lysozyme and the concentration of 1-ethyl-3-methyl imidazolium tetrafluoroborate;

FIG. 2: shows the relationship between the yield of renaturation for rPA and the concentration of 1-ethyl-3-methyl imidazolium tetrafluoroborate;

FIG. 3: shows the relationship between the yield of renaturation for rPA and the concentration of 1-ethyl-3-methyl imidazolium chloride;

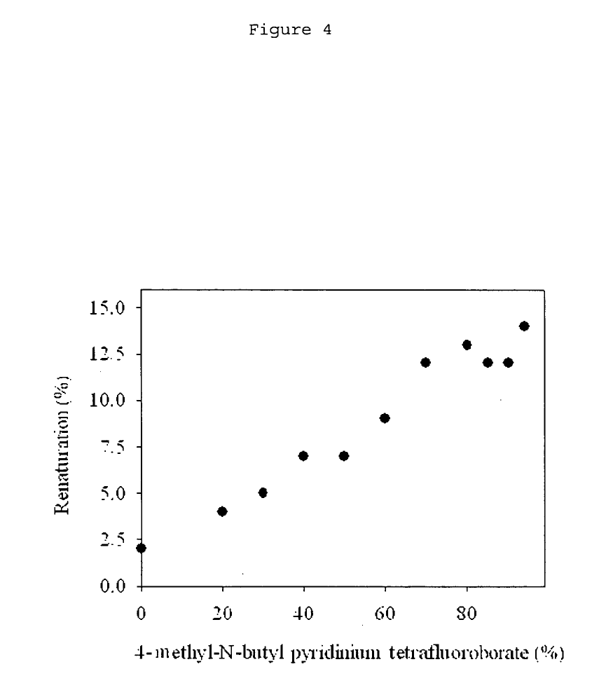

FIG. 4: shows the relationship between the yield of renaturation for rPA and the concentration of 4-methyl-N-butyl pyridinium tetrafluoroborate;

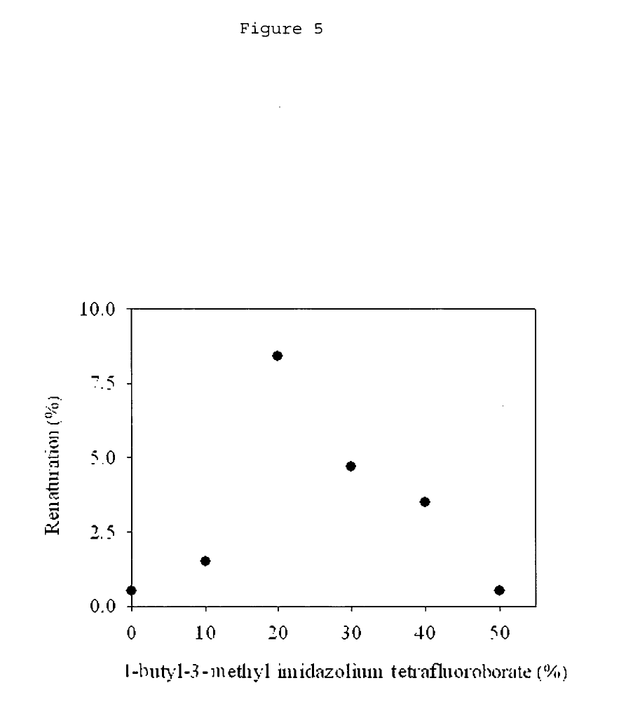

FIG. 5: shows the relationship between the yield of renaturation for rPA and the concentration of 1-butyl-3-methyl imidazolium tetrafluoroborate;

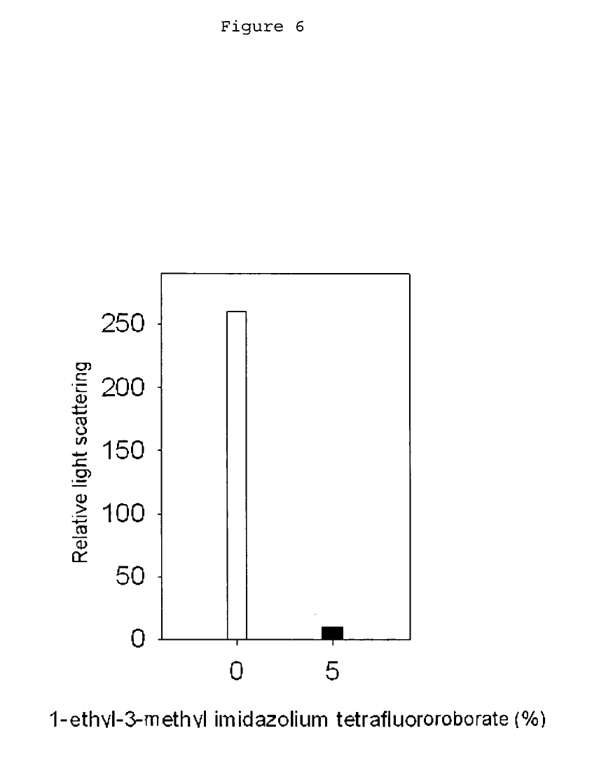

FIG. 6: is a graphic representation of the aggregation of rPA during renaturation as determined by means of light scattering wherein 0% and 5% 1-ethyl-3-methyl imidazolium tetrafluoroborate are contained;

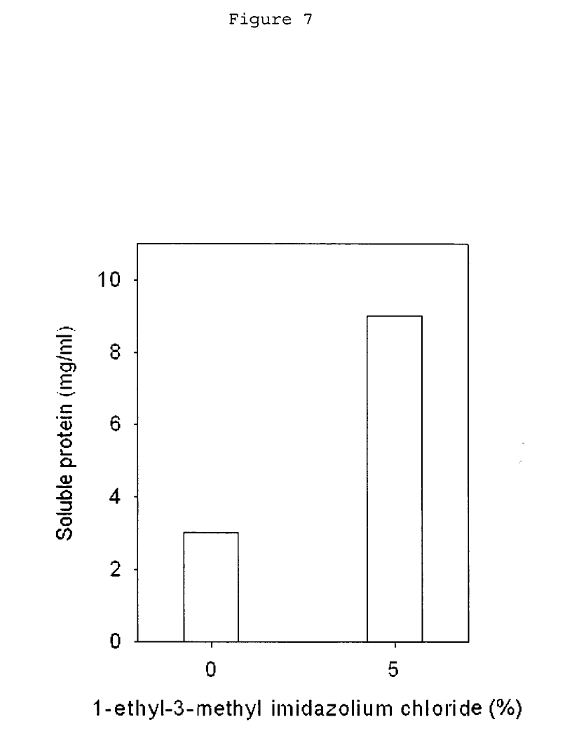

FIG. 7: shows the concentration of dissolved nGLP1R in the absence and in the presence of 5% 1-ethyl-3-methyl imidazolium chloride;

FIG. 8: is a graphic representation of the aggregation behavior of rPA in relation to the temperature and for different concentrations of 1-ethyl-3-methyl imidazolium tetrafluoroborate as determined by means of light scattering;

FIG. 9: shows the relationship between the yield of renaturation of rPA and the concentration of 3-(1-pyridinio)-1-propane sulfate;

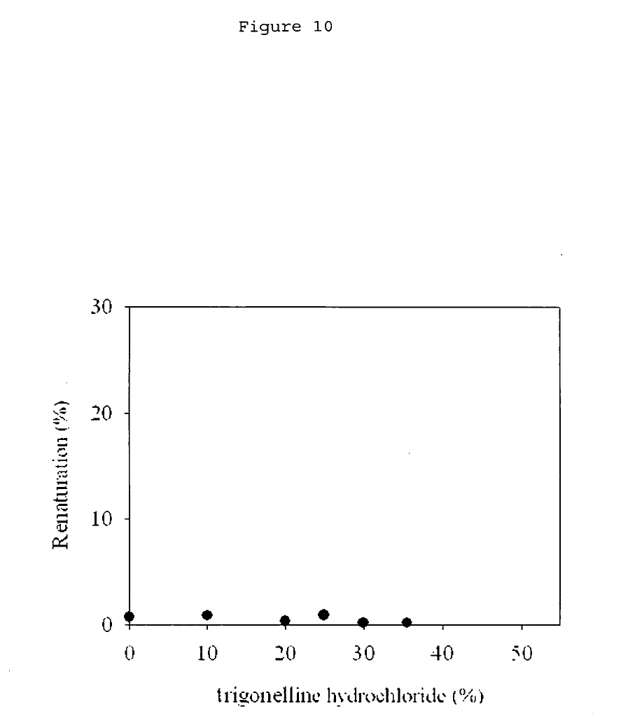

FIG. 10: shows the relationship between the yield of renaturation of rPA and the concentration of trigonelline hydrochloride.

### EXAMPLE 1

### Denaturation of Proteins

Lysozyme, rPA or inclusion body proteins at a protein concentration of 0.5-20 mg/ml in 0.1 M Tris, pH 8, 6 M guanidinium hydrochloride, 1 mM EDTA, 200 mM DTT were denatured and reduced for at least 2 h at room temperature. Subsequently, the pH value is lowered to about pH 2 by the addition of HCl. Optionally, the denatured protein can be dialyzed against 1000 times the volume of 6 M guanidinium hydrochloride, pH 2. The concentration of denatured protein is determined by means of the Bradford assay (Bradford, 1976) or spectrophotometrically. For this purpose, an extinction coefficient of e (1 mg/ml, 280 nm, 1 cm)=1 is used for rPA and an extinction coefficient of e (1 mg/ml, 280 nm, 1 cm)=2.37 is used for lysozyme.

### EXAMPLE 2

### Renaturation of Lysozyme

The renaturation of lysozyme is performed by an 1:100 dilution of the denatured, reduced protein into renaturation buffer which has been pre-equilibrated to 10° C. wherein the protein end concentration is 200 μg/ml. As the renaturation buffer there is used 0.05 M Tris, pH 8, 1 mM EDTA, 4 mM GSSG (oxidized glutathione) and different concentrations of the ionic liquid 1-ethyl-3-methyl imidazolium tetrafluoroborate. As the reducing agent serves 2 mM DTT which is carried over by dilution of the denaturation sample. After renaturation the samples are dialyzed against 1000 times the volume of 0.05 M Tris, pH 8, 1 mM EDTA over night. The enzymatic activity of lysozyme is measured photometrically and quantified with respect to a standard curve to provide a measure for the renaturation.

FIG. 1 shows the relationship between the yield of renaturation of lysozyme and the concentration of the salt 1-ethyl-3-methyl imidazolium tetrafluoroborate. In the presence of 10-15 vol. % of 1-ethyl-3-methyl imidazolium tetrafluoroborate there is quantitative, i.e. 100%, oxidative renaturation of lysozyme.

### EXAMPLE 3

### Renaturation of rPA Using 1-ethyl-3-methyl imidazolium tetrafluoroborate

The renaturation of rPA present in 6 M guanidinium, pH 2, is performed by an 1:100 dilution in 0.1 M Tris, pH 10.5, 1 mM EDTA, 5 mM GSSG, 2 mM GSH (reduced glutathione) in the presence of different concentrations of 1-ethyl-3-methyl imidazolium tetrafluoroborate. The renaturation is performed for at least 16 h at 10° C. After renaturation the samples are dialyzed against 1000 times the volume of 0.1 M Tris, pH 8, 1 mM EDTA over night. The enzymatic activity of rPA is determined by means of the Chromozyme tPA assay (Roche Diagnostics) and quantified with respect to a standard curve of native rPA to provide a measure for the renaturation.

FIG. 2 shows the relationship between the yield of renaturation of rPA and the concentration of the salt 1-ethyl-3-methyl imidazolium tetrafluoroborate. In the presence of 20-30 vol. % of 1-ethyl-3-methyl imidazolium tetrafluoroborate the yield of renaturation is 23%.

### EXAMPLE 4

### Renaturation of rPA Using 1-ethyl-3-methyl imidazolium chloride

The renaturation of rPA present in 6 M guanidinium, pH 2, is performed by an 1:100 dilution in 0.1 M Tris, pH 10.5, 1 mM EDTA, 5 mM GSSG, 2 mM GSH in the presence of different concentrations of 1-ethyl-3-methyl imidazolium chloride. The renaturation is performed for at least 16 h at 10° C. After renaturation the samples are dialyzed against 1000 times the volume of 0.1 M Tris, pH 8, 1 mM EDTA over night. The enzymatic activity of rPA is determined by means of the Chromozyme tPA assay (Roche Diagnostics) and quantified with respect to a standard curve of native rPA to provide a measure for the renaturation.

FIG. 3 shows the relationship between the yield of renaturation of rPA and the concentration of the salt 1-ethyl-3-methyl imidazolium chloride. In the presence of 20-25 vol. % of 1-ethyl-3-methyl imidazolium chloride the yield of renaturation is 26%.

### EXAMPLE 5

### Renaturation of rPA Using 4-methyl-N-butyl pyridinium tetrafluoroborate (Comparative Example)

The renaturation of rPA present in 6 M guanidinium, pH 2, is performed by an 1:100 dilution in 0.01 M Tris, pH 10.5, 1 mM EDTA, 5 mM GSSG, 2 mM GSH in the presence of different concentrations of 4-methyl-N-butyl pyridinium tetrafluoroborate. The renaturation is performed for at least 16 h at 10° C. After renaturation the samples are dialyzed against 100 times the volume of 0.1 M Tris, pH 8, 1 mM EDTA over night. The enzymatic activity of rPA is determined by means of the Chromozyme tPA assay (Roche Diagnostics) and quantified with respect to a standard curve of native rPA to provide a measure for the renaturation.

FIG. 4 shows the relationship between the yield of renaturation of rPA and the concentration of the salt 4-methyl-N-butyl pyridinium tetrafluoroborate. The yield of renaturation of rPA increases with increasing concentrations of 4-methyl-N-butyl pyridinium tetrafluoroborate. At a concentration of 94% (v/v) of 4-methyl-N-butyl pyridinium terafluoroborate the yield of renatured rPA is 13.5%.

### EXAMPLE 6

### Renaturation of rPA Using 1-butyl-3-methyl imidazolium tetrafluoroborate

The renaturation of rPA present in 6 M guanidinium, pH 2, is performed by an 1:100 dilution in 0.1 M Tris, pH 10.5, 1 mM EDTA, 5 mM GSSG, 2 mM GSH in the presence of different concentrations of 1-butyl-3-methyl imidazolium tetrafluoroborate. The renaturation is performed for at least 16 h at 10° C. After renaturation the samples are dialyzed against 1000 times the volume of 0.1 M Tris, pH 8, 1 mM EDTA over night. The enzymatic activity of rPA is determined by means of the Chromozyme tPA assay (Roche Diagnostics) and quantified with respect to a standard curve of native rPA to provide a measure for the renaturation.

FIG. 5 shows the relationship between the yield of renaturation of rPA and the concentration of the salt 1-butyl-3-methyl imidazolium tetrafluoroborate. In the presence of 20% (v/v) of 1-butyl-3-methyl imidazolium tetrafluoroborate the yield of renaturation is 8%.

### EXAMPLE 7

### Aggregation of rPA During the Renaturation

The renaturation of rPA present in 6 M guanidinium, pH 2, is performed by an 1:100 dilution in 0.1 M Tris, pH 10.5, 1 mM EDTA, 5 mM GSSG, 2 mM GSH in the presence or in the absence of 5% 1-ethyl-3-methyl imidazolium tetrafluoroborate. The renaturation is performed at 10° C. in a stirred fluorescence cuvette. During the kinetic the aggregation of the protein is monitored by measuring the light scattering by using excitation at 360 nm and emission at 360 nm.

FIG. 6 shows the buffer-corrected plateau value of the aggregation measurements of the renaturation of rPA in the absence of salt (open bar) and in the presence of 5 vol. % 1-ethyl-3-methyl imidazolium tetrafluoroborate (filled bar). In the presence of at least 5 vol. % 1-ethyl-3-methyl imidazolium tetrafluoroborate the aggregation of rPA to be renatured is almost completely suppressed.

### EXAMPLE 8

### Solubility of the N-terminal Domain of the GLP1 receptor (nGLP1R)

The native, structured receptor domain was incubated in 0.4 M potassium phosphate, pH 6.3, 0.4 M ammonium sulfate, 20° C. in the presence and in the absence of 5% l-ethyl-3-methyl imidazolium chloride at different protein concentrations over night. Subsequently, the protein aggregates were sedimented at 70,000 rpm and the fraction of the soluble protein was quantified by means of absorption spectrophotometry at 280 nm. FIG. 7 shows a comparison of the maximum concentrations of soluble protein which could be achieved under each of the conditions.

Thereby, this example shows the increase in solubility or the reduction of aggregation, respectively, by the substituted imidazolium salts.

### EXAMPLE 9

### Thermal Stability of rPA

Native rPA present in a concentration of 20 μg/ml in 0.1 M Tris, pH 10.5, 1 mM EDTA, 5 mM GSSG, 2 mM GSH in the presence of different concentrations of 1-ethyl-3-methyl imidazolium tetrafluoroborate is heated from 20° C. to 90° C. at a heating rate of about 0.3° C./min. During this process the aggregation state of the protein is analyzed by means of light scattering. For this purpose, the excitation of the sample is performed in a fluorimeter at 360 nm, and the signal is also detected at 360 nm.

FIG. 8 shows the aggregation behavior of rPA during thermal denaturation (filled circles 0 vol. %; filled triangles 5 vol. %; open circles 12 vol. %; open triangles 20 vol. % of 1-ethyl-3-methyl imidazolium tetrafluoroborate). The aggregation of rPA in the absence of 1-ethyl-3-methyl imidazolium tetrafluoroborate starts at 55° C. In the presence of 5 vol. % 1-ethyl-3-methyl imidazolium tetrafluoroborate an increased aggregation can only be observed at temperatures higher than 80° C. while no measurable aggregation up to 90° C. occurs at higher concentrations of 1-ethyl-3-methyl imidazolium tetrafluoroborate.

### EXAMPLE 10

### Renaturation of rPA Using 3-(1-pyridinio)-1-propane sulfate (Comparative Example)

The renaturation of rPA present in 6 M guanidinium, pH 2, is performed by an 1:100 dilution in 0.1 M Tris, pH 10.5, 1 mM EDTA, 5 mM GSSG, 2 mM GSH in the presence of different concentrations of 3-(1-pyridinio)-1-propane sulfate. The renaturation is performed for at least 16 h at 10° C. After renaturation the samples are dialyzed against 1000 times the volume of 0.1 M Tris, pH 8, 1 mM EDTA over night. The enzymatic activity of rPA is determined by means of the Chromozyme tPA assay (Roche Diagnostics) and quantified with respect to a standard curve of native rPA to provide a measure for the renaturation.

FIG. 9 shows the relationship between the yield of renaturation of rPA and the concentration of the salt 3-(1-pyridinio)-1-propane sulfate. In the presence of 30% (v/v) of 3-(1-pyridinio)-1-propane sulfate the maximum yield of renaturation is 18%.

### EXAMPLE 11

### Renaturation of rPA Using trigonelline hydrochloride (Comparative Example)

The renaturation of rPA present in 6 M guanidinium, pH 2, is performed by an 1:100 dilution in 0.1 M Tris, pH 10.5, 1 mM EDTA, 5 mM GSSG, 2 mM GSH in the presence of different concentrations of trigonelline hydrochloride. The renaturation is performed for at least 16 h at 10° C. After renaturation the samples are dialyzed against 1000 times the volume of 0.1 M Tris, pH 8, 1 mM EDTA over night. The enzymatic activity of rPA is determined by means of the Chromozyme tPA assay (Roche Diagnostics) and quantified with respect to a standard curve of native rPA to provide a measure for the renaturation.

FIG. 10 shows the relationship between the yield of renaturation of rPA and the concentration of the salt trigonelline hydrochloride. No improved renaturation of rPA is observed in the presence of the salt.

1. Use of substituted imidazolium salts for the renaturation, for the increase of the thermal stability and/or for the decrease of aggregation of proteins.

2. The use according to claim 1, wherein said imidazolium salts are substituted by alkyl, alkenyl, aryl and/or aralkyl groups which may themselves be substituted by functional groups, preferably by groups containing nitrogen, sulfur and/or phosphor.

3. The use according to claim 1, wherein said imidazolium salts are substituted at both nitrogen atoms of the imidazolium ring.

4. The use according to, claim 1, wherein said imidazolium salts are substituted at one or more of the carbon atoms of the imidazolium ring.

5. The use according to claim 1, wherein said imidazolium ring has C₁-C₄ alkyl groups, preferably methyl and/or ethyl groups, C₁-C₄ alkenyl groups, and/or mono- or bicyclic aryl groups as said substituents.

6. The use according to claim 1, wherein said imidazolium ring has a methyl group at at least one of the N atoms.

7. The use according to claim 1, wherein said imidazolium ring has one or more of the following substituents: phenol, biphenyl, biphenol, naphthalene, naphthalene carboxylic acids, naphthalene sulfonic acids, biphenylols, biphenyl carboxylic acids, phenol, phenyl sulfonate, phenol sulfonic acids and/or substituted or unsubstituted benzyl.

8. The use according to claim 1, wherein said substituents are further substituted by one or more of the following groups: amine, carboxyl, carbonyl, aldehyde, hydroxy, sulfate, sulfonate and/or phosphate groups.

9. The use according to claim 1, wherein said imidazolium salts are ionic liquids at room temperature.

10. The use according to claim 1 for the renaturation, the increase of the thermal stability and/or the decrease of the aggregation of:
proteases, preferably serine proteases, particularly thrombin, factor Xa, caspases, cathepsins, trypsin and/or chymotrypsin, cysteine proteases, acidic proteases such as pepsin or rennin, metalloproteinases such as thermolysin;
protease inhibitors, preferably pepstatins, antipain, chemostatins, elastinal, leupeptins, bestatin, antithrombin III;
DNA binding proteins, preferably transcription factors, particularly NF kappa B and members of the jun, fos, krox, myc, E2F families, and/or viral T antigens;
viral proteins such as viral envelope proteins, capsid proteins, viral proteases, polymerases and/or T antigens;
phosphatases;
protein kinases, preferably tyrosine kinases and/or serine kinases;
proteins of the immunoglobulin superfamily, e.g. antibodies and fragments thereof;
growth factors, preferably epidermal growth factor, erythropoietin, fibroblast growth factor, insulin-like growth factors I and II, interleukin-2, nerve growth factor, transforming growth factor β and/or thrombocyte growth factor;
lysozyme, rPA, alpha-glucosidase,
as well as proteins and fragments derived from these proteins.

11. A method for the renaturation, the increase of the thermal stability and/or the decrease of the aggregation of proteins, wherein the proteins to be treated are contacted with a liquid medium containing substituted imidazolium salts.

12. The method according to claim 11 for the renaturation of proteins wherein a liquid renaturation medium is used as the liquid medium.

13. The method according to claim 11, wherein a buffer system suitable for the protein to be treated is used as the liquid medium.

14. The method according to claim 11, wherein Tris, HEPES, Mes, Mops, acetate, glycine and/or phosphate are used as buffer substances.

15. The method according to claim 11, wherein the buffer concentration in the liquid medium is 10-1000 mM, preferably 5-200 mM, still more preferred 10-50 mM.

16. The method according to claim 11, wherein the pH value of the buffer system is 4-11, preferably 7.5-10.5.

17. The method according to claim 11, wherein said imidazolium salts are substituted by alkyl, alkenyl, aryl and/or aralkyl groups which may themselves be substituted by functional groups, preferably by groups containing nitrogen, sulfur and/or phosphor.

18. The method according to claim 11, wherein said imidazolium salts are substituted at both nitrogen atoms of the imidazolium ring.

19. The method according to claim 11, wherein said imidazolium salts are substituted at one or more of the carbon atoms of the imidazolium ring.

20. The method according to claim 11, wherein said imidazolium ring has C₁-C₄ alkyl groups, preferably methyl and/or ethyl groups, C₁-C₄ alkenyl groups, and/or mono- or bicyclic aryl groups as said substituents.

21. The method according to claim 11, wherein said imidazolium ring has a methyl group at at least one of the N atoms.

22. The method according to claim 11, wherein said imidazolium ring has one or more of the following substituents: phenol, biphenyl, biphenol, naphthalene, naphthalene carboxylic acids, naphthalene sulfonic acids, biphenylols, biphenyl carboxylic acids, phenol, phenyl sulfonate, phenol sulfonic acids and/or substituted or unsubstituted benzyl.

23. The method according to claim 11, wherein said substituents are further substituted by one or more of the following groups: amine, carboxyl, carbonyl, aldehyde, hydroxy, sulfate, sulfonate and/or phosphate groups.

24. The method according to claim 11, wherein said imidazolium salts are ionic liquids at room temperature.

25. The method according to claim 11, wherein the contacting is performed by diluting, dialyzing and/or diafiltrating the protein to be treated with the liquid medium.

26. The method according to claim 11, wherein the protein concentration of the protein to be treated during contacting is 5-500 μg/ml, preferably 10-200 μg/ml, still more preferably about 50-100 μg/ml.

27. The method according to claim 11, wherein the concentration of substituted imidazolium salts is 5-95 vol. %, preferably 5-50 vol. %, most preferred 10-40 vol. % based on the liquid medium.

28. The method according to claim 11, wherein the molarity of substituted imidazolium salts is 0.25-5 M, preferably 0.25-3.5 M, most preferably 0.5-3 M based on the liquid medium.

29. The method according to claim 11, wherein the method is performed by adding the protein to be renatured to the renaturation medium at several successive times or in a continuous manner.

30. The method according to claim 11, wherein the contacting of the proteins to be renatured with the renaturation medium is performed for 0.1-100 h, preferably 1-50 h, still more preferred 5-20 h.

31. The method according to claim 11 for the renaturation, the increase of the thermal stability and/or the decrease of the aggregation of:
proteases, preferably serine proteases, particularly thrombin, factor Xa, caspases, cathepsins, trypsin and/or chymotrypsin, cysteine proteases, acidic proteases such as pepsin or rennin, metalloproteases such as thermolysin;
protease inhibitors, preferably pepstatins, antipain, chemostatins, elastinal, leupeptins, bestatin, antithrombin III;
DNA binding proteins, preferably transcription factors, particularly NF kappa B and members of the jun, fos, krox, myc, E2F families, viral T antigens;
viral proteins such as viral envelope proteins, capsid proteins, viral proteases, polymerases and/or T antigens;
phosphatases;
protein kinases, preferably tyrosine kinases and/or serine kinases;
proteins of the immunoglobulin superfamily, e.g. antibodies and fragments thereof;
growth factors, preferably epidermal growth factor, erythropoietin, fibroblast growth factor, insulin-like growth factors I and II, interleukin-2, nerve growth factor, transforming growth factor β and thrombocyte growth factor;
lysozyme, rPA, alpha-glucosidase,
as well as proteins and fragments derived from these proteins.

32. The method according to claim 1, wherein disulfide-free proteins are renatured wherein the contacting of the disulfide-free protein with the renaturation medium is performed in the presence of a reducing agent, preferably DTT, DTE and/or cysteine.

33. The method according to claim 11, wherein disulfide-bridged proteins are renatured wherein the contacting of said disulfide-bridged protein with the renaturation medium is performed in the presence of a redox system composed of reduced and oxidized thiol substances such as DTT, DTE, glutathione, cysteine, and/or mercaptoethanol.

34. The method according to claim 32, wherein the concentration ratio of reduced substances to oxidized substances is 1:10 to 20:1, preferably 1:5 to 10:1, further preferred 1:1 to 5:1.

35. The method according to claim 11, wherein the liquid medium for the renaturation of proteins contains other renaturation substances, preferably urea, guanidinium, L-arginine, alkyl urea, carboxylic acid amides, alkylated amines, Tris buffers, polyethylene glycol and/or detergents.

36. (Deleted)
